(19) United States
(12) Patent Application Publication
(10) Pub. No.: US 2023/0260616 A1
(43) Pub. Date: Aug. 17, 2023

(54) USER DEVICE COMMUNICATION DEVICE

(71) Applicant: Modular Medical , Inc., San Diego, CA (US)

(72) Inventors: Paul M. DiPerna, San Diego, CA (US); Marc D. Goldman, San Diego, CA (US); Kelsie DiPerna, San Diego, CA (US)

(21) Appl. No.: 17/673,124

(22) Filed: Feb. 16, 2022

(51) Int. Cl. G16H 20/17; G08B 3/10; G08B 5/36; G08B 6/00

(52) U.S. Cl. CPC G16H 20/17; G08B 3/10; G08B 5/36; G08B 6/00

(57) ABSTRACT

A system for controlling the operations of a computerized device requires a sequence of two-way communications between a user and a computer. These communications both validate and control the operation of the device. A first communication from the user to the computer provides instructions that typically include changes for an operation of the device. In response, the computer requests user validation and identification. A second communication from the computer to the user then provides an operational status report of the system and a request for operational confirmation from the user, together with revalidation of user identification. An operation of the communication system is accomplished by selectively using audio, visual, and tactile signals from the user, interactively with vibrations, light variations, visual presentations and sound sequences from the device.

### FIELD OF THE INVENTION

The present invention pertains to systems and methods for controlling computerized devices. More particularly, the present invention pertains to communication techniques and procedures for controlling the operation of devices, such as an infusion pump, which must function in accordance with a predetermined clinical protocol for regulatory compliance. The present invention is particularly, but not exclusively useful as a communication system for employing a combination of aural, visual and tactile means to execute and validate the instructions needed for controlling a computerized device.

### BACKGROUND OF THE INVENTION

The use of medical devices that are to be operated and controlled by an end-user, without the supervision of trained medical personnel, raises several issues that are not otherwise particularly relevant. An underlying premise here is that the user is both competent and capable of properly operating the device. Although this may initially be a valid presumption, for any of several reasons this may change.

A particular concern for self-operated medical devices is that their operation must comply with regulatory requirements, and they must somehow be properly monitored. Specifically, when a medical protocol has been prescribed, it is essential that the device be operated in appropriate compliance with the protocol. For instance, when the medical device is an infusion pump, and infusions are to be performed in accordance with a prescribed protocol, deviations from the protocol are unacceptable. The situation can become even more problematical when an unqualified caregiver becomes involved.

To safeguard against a potentially dangerous operation of a self-operated Medical device, it is important for any temporal and/or dosage changes to a medical infusion protocol be made in compliance with the protocol's requirements. Moreover, it is also important that any changes be made by the user himself/herself or by trained personnel who are authorized to make the appropriate change.

With the above in mind, it is an object of the present invention to provide a communication system for operating a computer-controlled medical device that provides for two-way communications between the user and the computer controlling an operation of the device. Another object of the present invention is to establish two-way communication between a medical device and the user of the device, whereby the computer's operation is dependent on the validated confirmation of instructions from an appropriately identified user. Still another object of the present invention is to provide a self-controlled medical device which can only be operated by an identified user with regulatory compliance in accordance with a prescribed medical protocol. Yet another object of the present invention is to provide a communication system for operating a computer-controlled medical device that is easy to use, is simple to manufacture and is competitively cost effective.

### SUMMARY OF THE INVENTION

A communication system for operating a computer-controlled device in accordance with real time, updated instructions from a user requires a computer that is directly connected with the device. An interface unit which is adapted for communication applications with the user is also mounted on the computer. Specifically, the communication applications provided by the interface unit allow for various combinations of audio, visual, and tactile modes of signal transmissions between a live, animate entity (i.e. a human user) and an inanimate entity (i.e. a computer).

Included in the interface unit on the computer is a transceiver with a receiver for receiving instructions from the user in the transmission of a first communication. Specifically, the first communication will include instructions from the user to the computer, for a specified operation of the computer-controlled device. In response to the first communication from the user, a request is transmitted by the computer back to the user for an operational confirmation of the instructions that are received from the user. Together with this request, the computer will provide a first prearranged signal for validating a subsequent identification of the user.

In response to the first communication, the transmitter of the transceiver in the interface unit on the computer is used for initiating a second communication between the computer and the user. Specifically, the second communication includes an advisory statement from the computer to the user regarding the operational status of the computerized system. It also includes a request for reconfirming the response from the user together with a second prearranged signal for identifying the user. The user then responds with the requested reconfirmation and an order to execute the instructions.

As envisioned for the present invention, the first communication is accomplished by the user in a manner selected from the group consisting of audio, visual, and tactile signals from the user. A response and the second communication are accomplished by the computer in a manner selected from the group consisting of vibrations, light variations, visual presentations, and sound sequences. As will be appreciated by a person of ordinary skill, there are many modes of signal transmissions that may be adapted for use with communication applications of the present invention. The important characteristic of communications between the user and the computer is that they are conversational in the sense that cross verifications, validations, confirmations, and identifications that are unique to the user and the computer are employed for controlling an operation of the device.

For a preferred embodiment of the present invention, the computer-controlled device is an infusion pump. Accordingly, instructions from the user in the first communication will typically include time, dosage and real time updates for medication changes that are compliant with a protocol that has been clinically incorporated for use in the computer. Stated differently, the execution and functional control of the computer-controlled device by the computer must comply with the protocol. To support a flexible functionality of the protocol, a monitor can be connected to the user for collecting data that is pertinent to a physical characteristic or the user (e.g., blood glucose levels). This data can then be used for compliant execution and control of the protocol by the computer. However, in the event an anomaly is detected by the monitor, the computer-controlled device executes an alarm message indicating that the collected data is noncompliant with the protocol. The alarm message is transmitted immediately to both the user, and to designated clinical personnel.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 illustrates the communications channels envisioned for the present invention, which provide for real time, updated instructions from an animate human user to an inanimate computer for operating a computer-controlled device;

FIG. 2 is a schematic presentation of components for a computer that is used to control the operation of a device in accordance with the present invention;

FIG. 3 shows exemplary contents for communications applications needed to establish computer control over the operation of a remote device; and

FIG. 4 shows exemplary signal transmission modes used for communications applications of the present invention.

### DESCRIPTION OF THE PREFERRED EMBODIMENTS

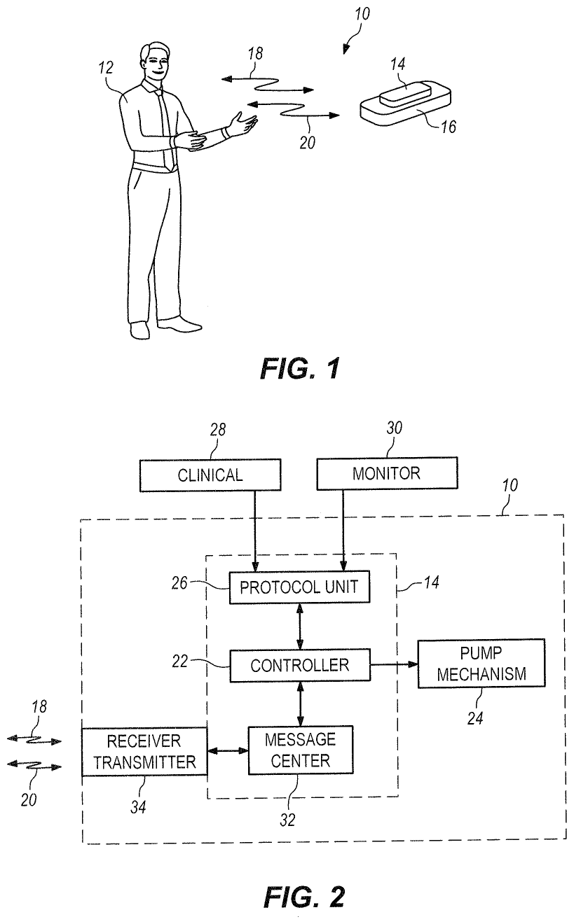

Referring initially to FIG. 1 a communication system for remotely controlling the operation of a device is shown and is generally designated 10. As shown, the system 10 involves a user 12 that remotely interacts directly with a computer 14, via a plurality of different modes of communication. The intended purpose of this communication is to provide instructions for the operation of a device 16 which is controlled by the computer 14. In a preferred embodiment of the system 10, the device 16 will be an infusion pump which is adapted for use by the user 12 for medical purposes.

As shown in FIG. 1, the communication between the user 12 and the computer 14 is essentially a bifurcated conversation that includes a first communication 18 and a subsequent, follow-up, second communication 20. Generally, in the first communication 18, the user 12 provides instructions to the computer 14 for an updated operation of the device 16 (i.e., a change order). The second communication 20 is then undertaken for the purposes of: i) confirming the operational instructions; ii) validating the identity of the user 12; iii) providing the user 12 with an advisory statement regarding the operational status of the system 10; and iv) verifying the operational efficacy of the intended result. The user 12 then sends the order to execute the instructions for an operation of the device 16.

FIG. 2 shows that the computer 14 includes a controller 22 which is connected to a pumping mechanism 24 in the device 16. Specifically, as intended for the system 10, the controller 22 will control the pumping mechanism 24 of the device 16 in accordance with operational parameters provided by a protocol unit 26. Input for the protocol unit 26 includes clinical information 28 that is pertinent to the medical condition of the user 12. This clinical information 28 may also include updated information, in real time, that is collected by a monitor 30 that may be affecting the medical condition of the user 12.

FIG. 2 also shows that the computer 14 includes a message center 32 which interconnects the controller 22 with an interface unit 34. For purposes of the present invention, the interface unit 34 is a transceiver of a type well known in the pertinent art that includes both a receiver and a transmitter. As shown, the interface unit 34 establishes the communications link between the computer 14 and the user 12 for both the first communication 18 and the second communication 20.

A method for manufacturing a communication system 10 that remotely controls a computerized device 16 requires mounting a plurality of diverse electronic components on the computer 14. This includes installing a message center 32 on a base structure of the computer 14 for transmitting and receiving interactively validated communications between the user 12 of the system 10 and the computer 14. Also, it is necessary to establish a protocol unit 26 on the computer 14 for receiving clinical information 28 that defines the predetermined procedure of a protocol for operating the device 16. Functionally, the protocol unit 26 is established to govern implementation of interactive communications between the user 12 and the computerized device 16.

The manufacture of a system 10 for the present invention further requires locating the controller 22 on the computer 14 where it can be electronically connected between the protocol unit 26 and the message center 32. In this combination, the controller 22 operates the computerized device 16 in accordance with the predetermined procedure established for the protocol unit 26. Specifically, for the present invention, this results in implementing the validated and confirmed content in the first communication 18 and the second communication 20 as they are received and transmitted between the message center 32 and the user 12.

An important aspect of the system 10 for the present invention is how communications are passed between the user 12 and the computer 14. For this purpose, the interface unit 34 on the computer 14 is a transceiver having a receiver for receiving instructions from the user 12 that are presented in the transmission of the first communication 18. Specifically, the first communication 18 will include instructions from the user 12 to the computer 14 for a specified operation of the computer-controlled device 16. In response to the first communication 18 from the user 12, a request is transmitted by the computer 14 from the message center 32 hack to the user 12 for an operational confirmation of the instructions sent from the user 12. Together with this request, the computer 14 will also provide a first prearranged signal for validating a subsequent identification of the user 12.

In response to the first communication, the transmitter of the transceiver in the interface unit 34 is used for initiating the second communication 20 between the computer 14 and the user 12. Specifically, the second communication 20 includes an advisory statement from the computer 14 for the user 12 regarding the operational status of the computerized system 10. This second communication 20 will also include a request for reconfirming the response from the user 12 together with a second prearranged signal for identifying the user 12. The user 12 then responds with the requested reconfirmation and an order to execute the instructions.

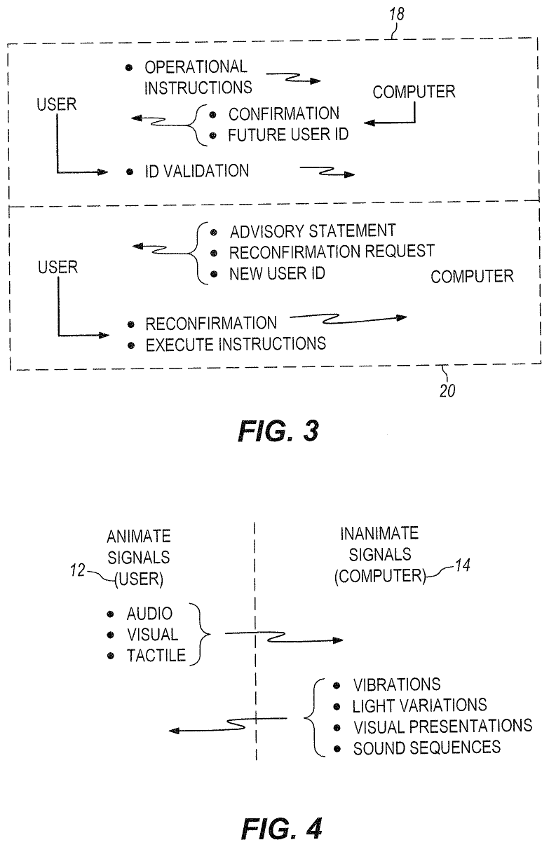

FIG. 3 illustrates the temporal context for an exchange of the general contents in both the first communication 18 and the second communication 20. In this exchange, it is to be appreciated that the user 12 will initiate the first communication 18, while it is the computer 14 that initiates the second communication 20. Moreover, unlike the instructional function of the first communication 18, the second communication 20 provides an advisory statement that is based on an evaluation of requirements from the protocol unit 26 regarding the operational efficacy of instructions contained in the first communication 18.

FIG. 4 illustrates the various modes for transmitting signals between the user 12 and the computer 14. In a preferred embodiment of the system 10 the user 12, who is an animate entity, will typically employ audio, visual, and/or tactile signals for his/her communication to the computer 14. On the other hand, the computer 14, which is an inanimate entity, will typically employ vibrations, light variations, visual presentations and/or sound sequences from the device 16 for its communication with the user 12.

While the particular User/Device Communication System as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A non-transitory, computer-readable medium having executable instructions stored thereon that direct a computer system to perform a process for connecting a user in communication with a computerized device which comprises:
a computer controlled by the medium;
instructions for a first communication between the user and the computer, initiated by the user of the device, wherein the first communication includes instructions from the user to the computer for a specified operation of the computerized device, and further wherein a request is made from the computer back to the user for a validating response from the user together with a first prearranged signal for identifying the user; and
instructions for a second communication, initiated by the computer in response to the first communication, wherein the second communication includes an advisory statement from the computer to the user regarding an operational status of the computerized system, with a request for an operational confirmation from the user together with a second prearranged signal for identifying the user.

2. The medium of claim 1 wherein the computerized device is an infusion pump, and instructions from the user include time, dosage and medication changes for a protocol clinically established in the medium for execution and control by the computer.

3. The medium of claim 2 wherein the first communication is initiated, and the instructions and validating response from the user are accomplished, in a manner selected from the group consisting of audio, visual, and tactile signals from the user.

4. The medium of claim 2 wherein the second communication is initiated, and the advisory statement and request for a revalidating response are accomplished in a manner selected from the group consisting of vibrations, light variations, visual presentations and sound sequences from the device.

5. The medium of claim 2 wherein the computerized device further comprises a monitor connected with the user for collecting data pertinent to a physical characteristic of the user in accordance with the protocol for execution and control by the computer.

6. The medium of claim 5 wherein the computerized device executes an alarm message when data collected by the monitor is noncompliant with the protocol.

7. The medium of claim 6 wherein the alarm message is transmitted immediately to the user and to designated clinical personnel.

8. The medium of claim 1 wherein the first communication and the second communication are initiated independently of one another.

9. A communication system for operating a computer-controlled device in accordance with instructions from a user which comprises:
a computer connected with the device;
an interface unit mounted on the computer and adapted for communication applications with the user;
a receiver included in the interface unit for receiving instructions for a first communication between the user and the computer, initiated by the user of the device, wherein the first communication includes instructions from the user to the computer for a specified operation of the computerized device, and further wherein a request is made from the computer back to the user far an operational confirmation from the user together with a first prearranged signal for identifying the user; and
a transmitter included in the interface unit for transmitting instructions for a second communication, initiated by the computer in response to the first communication, wherein the second communication includes an advisory statement from the computer to the user regarding an operational status of the computerized system, with a request for a revalidating response from the user together with a second prearranged signal for identifying the user.

10. The system of claim 9 wherein the computer-controlled device is an infusion pump, and instructions from the user include time, dosage and medication changes for a protocol clinically established in the computer for execution and control by the computer.

11. The system of claim 10 wherein the first communication is initiated, and the instructions and validating response from the user are accomplished, in a manner selected from the group consisting of audio, visual, and tactile signals from the user, and wherein the second communication is initiated, and the advisory statement and request for a revalidating response are accomplished in a manner selected from the group consisting of vibrations, light variations, visual presentations and sound sequences from the device.

12. The system of claim 11 wherein the computerized device further comprises a monitor connected with the user for collecting data pertinent to a physical characteristic of the user in accordance with the protocol for execution and control by the computer, and wherein the computer-controlled device executes an alarm message when data collected by the monitor is noncompliant with the protocol.

13. The system of claim 12 wherein the alarm message is transmitted immediately to the user and to designated clinical personnel.

14. The system of claim 13 wherein the first communication and the second communication are initiated independently of one another.

15. A method for manufacturing a computer interface for controlling a computerized device which comprises the steps of:
providing a base structure for supporting a plurality of electronic components of the computer;
connecting the computerized device with at least one electronic component of the computer on the base structure;
installing a message center on the base structure for transmitting and receiving interactively validated communications respectively to and from a user of the computerized device;
establishing a protocol unit on the base structure for maintaining a predetermined procedure to govern implementation of operational content in the interactive communications between the user and the computerized device; and
locating a controller on the base structure, wherein the controller is electronically connected with the computerized device, with the protocol unit and with the message center, wherein the controller operates the computerized device in accordance with the predetermined procedure of the protocol unit by implementing validated and confirmed content in the communications received and transmitted between the message center and the user.

16. The method of claim 15 further comprising the steps of:
creating the message center for accomplishing the first communication with electronic components selected from the group consisting of audio, visual, and tactile signals; and
constructing the message center for accomplishing the second communication with electronic components selected from the group consisting of vibrations, light variations, visual presentations and sound sequences from the device.

17. The method of claim 16 further comprising the steps of:
monitoring a collection of data pertinent to a physical characteristic of the user in accordance with the protocol for execution and control by the computer; and
executing an alarm message when data collected by the monitor is noncompliant with the protocol.

18. The method of claim 17 wherein the computerized device is an infusion pump, and instructions from the user include time, dosage and medication changes for a protocol clinically established for execution and control by the computer.

19. The method of claim 18 wherein the alarm message is transmitted immediately to the user and to designated clinical personnel.

20. The method of claim 19 wherein the first communication and the second communication are initiated independently of one another.
